(19) United States
(12) Patent Application Publication
(10) Pub. No.: US 2021/0341418 A1
(43) Pub. Date: Nov. 4, 2021

(54) DETECTION OF OXIDANT IN SEAWATER

(71) Applicant: Hach Company, Loveland, CO (US)

(72) Inventors: Corey Alan Salzer, Fort Collins, CO (US); Ralf Leidner, Fort Collins, CO (US)

(21) Appl. No.: 16/864,675

(22) Filed: May 1, 2020

(51) Int. Cl. G01N 27/416; G01N 27/48; G01N 27/30; G01N 27/38

(52) U.S. Cl. CPC G01N 27/4166; G01N 27/38; G01N 27/308; G01N 27/48

(57) ABSTRACT

An embodiment provides a method for measuring total oxidant in a seawater sample, comprising: forming a seawater solution and a formed iodine by introducing a buffer and an iodide reagent to a seawater sample, wherein the seawater sample contains an amount of oxidant; placing the seawater solution in a measurement device, wherein the measurement device comprises a boron doped diamond working electrode reacting with the seawater solution and the formed iodine, wherein an electrochemical process reduces the formed iodine to iodide; and measuring the amount of total oxidant in the seawater sample by measuring, using the measurement device, an amount of iodide in the seawater sample. Other aspects are described and claimed.

### BACKGROUND

This application relates generally to measuring oxidant in aqueous liquid samples, and, more particularly, to the measurement of oxidant in seawater using an electrochemical technique.

Ensuring water quality is critical in a number of industries such as pharmaceuticals, manufacturing fields, food preparation, or the like. Additionally, ensuring water quality is critical to the health and well-being of humans, animals, and plants which are reliant on the water for survival. One component that is typically measured is oxidant. Oxidant measurement may be a used as a measurement of chlorine in a water sample used for water treatment. Proper measurement of a disinfection agent may be critical to water treatment. Too much chlorine in water can be harmful to humans, animals, and aquatic life. Therefore, detecting the presence and concentration of chlorine in seawater, water, ballast water, or other liquid solutions is vital.

### BRIEF SUMMARY

In summary, one embodiment provides a method for measuring total oxidant in a seawater sample, comprising: forming a seawater solution and a formed iodine by introducing a buffer and an iodide reagent to a seawater sample, wherein the seawater sample contains an amount of oxidant; placing the seawater solution in a measurement device, wherein the measurement device comprises a boron doped diamond working electrode reacting with the seawater solution and the formed iodine, wherein an electrochemical process reduces the formed iodine to iodide; and measuring the amount of total oxidant in the seawater sample by measuring, using the measurement device, an amount of iodide in the seawater sample.

Another embodiment provides a measurement device for measuring total oxidant in a seawater sample, comprising: at least one measurement chamber; a processor; and a memory storing instructions executable by the processor to: form a seawater solution and a formed iodine by introducing a buffer and an iodide reagent to a seawater sample, wherein the seawater sample contains an amount of oxidant; place the seawater solution in a measurement device, wherein the measurement device comprises a boron doped diamond working electrode reacting with the seawater solution and the formed iodine, wherein an electrochemical process reduces the formed iodine to iodide; and measure the amount of total oxidant in the seawater sample by measuring, using the measurement device, an amount of iodide in the seawater sample.

A further embodiment provides a product for measuring total oxidant in a seawater sample, comprising: a storage device having code stored therewith, the code being executable by the processor and comprising: code that forms a seawater solution and a formed iodine by introducing a buffer and an iodide reagent to a seawater sample, wherein the seawater sample contains an amount of oxidant; code that places the seawater solution in a measurement device, wherein the measurement device comprises a boron doped diamond working electrode reacting with the seawater solution and the formed iodine, wherein an electrochemical process reduces the formed iodine to iodide; and code that measures the amount of total oxidant in the seawater sample by measuring, using the measurement device, an amount of iodide in the seawater sample.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a flow diagram of an example total oxidant measuring system for a seawater sample.

FIG. 2 illustrates an example total oxidant measuring device using a BDD working electrode.

FIG. 3 illustrates an example measurement method of total oxidant using square wave voltammetry.

FIG. 4 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

Oxidative reduction potential (ORP) may be used to measure an ability of a component of a water sample to oxidize or reduce another substance. A measurement may be made electrochemically upon a water sample to determine an ORP value for a given sample component. ORP may be a used as a method to determine the effectiveness or amount of an agent used for the disinfection of water. An example of such an agent may be chlorine. ORP electrodes commonly employed for measurement of oxidants are often comprised of platinum or gold electrodes. ORP electrodes can suffer from surface fouling and/or oxidation changes at their surface which may adversely impact measurement accuracy and require frequent maintenance to clean and recalibrate.

Chlorine measurement may be used to determine the quality of water. High concentrations of chlorine may be harmful to animals, humans, plants, and/or water ecosystems. As another example, a facility may want the chlorine in a body of water to be under a particular threshold, therefore, the user may measure the chlorine in order to determine if the amount of chlorine is under that threshold. For example, chlorine may need to be below a regulated threshold in ballast water prior to retuning the ballast water to a port environment.

A standard analytical reagent for free and total chlorine measurement in water is DPD (N,N-diethyl-p-phenylenediamine) via colorimetric detection. Total chlorine is the sum of free and combined chlorine in the sample and may include chlorine that has reacted with nitrogen compounds in the water. In the absence of iodide ion, free chlorine reacts quickly with DPD indicator to produce a red color, whereas combined chlorine species, such as chloramines, may react more slowly. If a small amount of iodide ion is added, combined chlorines, such as chloramines, may react with iodide to form iodine which quickly reacts with DPD to produce color, yielding total chlorine concentration. Absorbance (for example, at 515 nm) may be spectrophotometrically measured and compared to a series of standards, using a graph or a regression analysis calculation to determine free and/or total chlorine concentration.

There are disadvantages and problems associated with conventional colorimetric DPD-based analyzers. These issues may be a problem when measuring total residual oxidants (TRO) in a range of water types, but may be most problematic when testing seawater. Reagents associated with DPD-based total oxidant measurement may degrade over time. Thus, frequent changing of reagents may be necessary. These reagent changes cost a facility both time and money for labor and reagent cost. Additionally, DPD reagent can stain or discolor measurement equipment over time requiring cleaning and/or replacement. What is needed is a system and method to measure total oxidant accurately in seawater and reduce maintenance cycle and improve reagent stability.

Accordingly, an embodiment provides a system and method for measuring total oxidant or total residual oxidant (TRO) in a seawater sample. The seawater sample may be drawn from a ballast system in a ship. For example, ballast water may be treated with a chlorine-based method, and the total oxidant levels may need to be returned to low enough levels prior to de-ballasting the water back into the environment. The total oxidant in the ballast water may be total chlorine. In an embodiment, the method may obtain a seawater sample. The seawater sample may form a solution and a formed iodine by introducing a buffer and an iodide reagent to the seawater sample. The iodide reagent may be potassium iodide. The buffer may be an acid, such as hydrochloric acid. In an embodiment, the seawater solution may be placed in a measurement device. The measurement device may be a three-electrode electrochemical cell. In an embodiment a boron doped diamond (BDD) electrode may be used as a working electrode. The measurement device may measure an electrochemical characteristic of the seawater sample. The formed iodine may be measured by an electrochemical reduction. In an embodiment, the total oxidant may be proportional to a current produced by the measurement device through an electrochemical reaction of the iodine in the seawater sample and the BDD working electrode. In an embodiment, the total oxidant may be measured using square wave voltammetric methods. In an embodiment, a measurement chamber may have a cleaning device or a cleaning cycle. The cleaning may be accomplished using electrochemical or mechanical methods.

In an embodiment, the system may apply an electrical signal to the volume of aqueous solution in a chamber. The electrical signal may be applied using one or more electrodes, for example, a series of electrodes. Electrodes may include a working electrode, counter (auxiliary) electrode, reference electrode, or the like. In an embodiment, the one or more series of electrodes may be boron doped diamond (BDD) electrodes. The use of BDD serves as a better electrode material than other carbon-based or metallic materials (e.g., silver, gold, mercury, nickel, etc.) because these materials may eventually themselves become oxidized, thereby generating interfering signals and contributing to the errors in the measurement. The use of boron doped working electrode may allow for lower interferences and background current, thus providing for a more accurate measurement as compared to metal electrodes. BDD electrodes have lower fouling issues compared with other electrode materials. Using a BDD electrode in voltammetric and/or amperometric methods may provide a more sensitive and stable system than conventional methods such as colorimetric and ORP/I-based systems.

The illustrated example embodiments will be best understood by reference to the figures. The following description is intended only by way of example, and simply illustrates certain example embodiments.

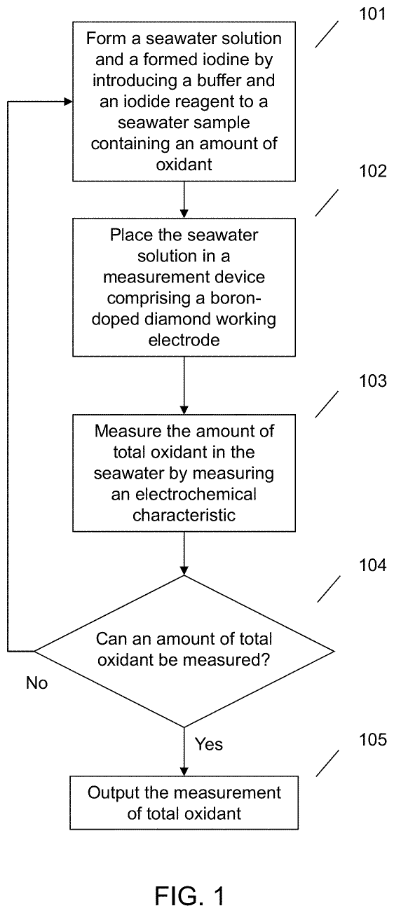

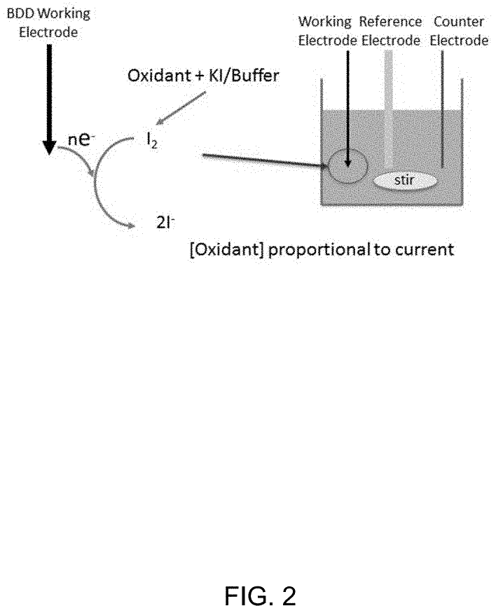

Referring to FIG. 1, an example system and method for detection of total oxidant in a seawater sample is illustrated. The seawater sample may be obtained from a ship ballast system. In an embodiment, a measurement device may comprise a three-electrode electrochemical cell (See FIG. 2). The working electrode may be a BDD electrode. In an embodiment, the method and system may use a voltammetric/amperometric approach to measure total oxidant. In an embodiment, the BDD electrode may provide operational longevity, wide measurement ranges, and/or diagnostics. In an embodiment, seawater of a known volume may be obtained and placed in a sample cell. The sample cell may have electrodes. The electrodes may comprise a working, auxiliary, and reference electrode. A buffer may be added to the sample. Potassium iodide may be added to the sample. The sample may be stirred. In an embodiment, oxidant in the seawater sample may react with iodide to form iodine (See FIG. 2). The formed iodine may be measured electrochemically and be related to an oxidant concentration. The formed iodine may be electrochemically reduced at the working electrode. The response from the electrochemical reduction response may be compared to a first background response. An ORP and a amperometric/voltammetric electrochemical measurements may be used independently or in combination to determine the TRO of a seawater sample.

At 101, in an embodiment, a seawater solution and an iodine may be formed. In an embodiment, a sample of seawater may be obtained from a ship ballast system. The seawater sample may be of a known volume. The known volume may be used to calculate proper reagent amounts to be added to the sample. The seawater sample may be pumped, gravity fed, or the like from a ship ballast tank, disinfection unit, or any portion of the ballast system. The sample may be of seawater that is to undergo treatment or has already undergone a treatment. For example, a measurement of oxidant may be made prior to a disinfection treatment. Additionally, or alternatively, an oxidant measurement may be performed after the addition of chlorine for treatment, but before the seawater is discharge overboard to ensure oxidant levels are sufficient for disinfection or levels low enough for discharge.

In an embodiment, an iodide reagent may be added to the seawater sample. The iodide may be, for example, potassium iodide. In an embodiment a buffer may be added to the seawater sample. The buffer may be an acid buffer reagent. The acid buffer may be hydrochloric acid. In an embodiment, the iodide and buffer reagent added to the sample reacts with oxidant in the sample to form iodine. In an embodiment, the reduction of iodine and/or triiodide formed by the reaction of iodide with oxidants in a seawater sample may be achieved by electrochemical reduction.

The seawater sample which may include a sample from a natural body of water, a holding tank, a processing tank, a pipe, a ballast water system, a ballast water treatment system, sea chest, ballast tank, or the like. The solution may be in a continuous flow, a standing volume of liquid, or any combination thereof. Introduction of the seawater sample into the measurement device may include placing or introducing the seawater sample into a test chamber manually by a user or using a mechanical means, for example, gravity flow, a pump, pressure, fluid flow, or the like. For example, a water sample for chlorine testing may be introduced to a measurement or test chamber using a pump. In an embodiment, valves or the like may control the influx and efflux of the solution into or out of the one or more chambers, if present.

At 102, in an embodiment the seawater solution and the formed iodine may be placed in a measurement device. The measurement device may have a sample or measurement cell to receive the sample. In an embodiment, the measurement cell may be the same vessel or a different vessel in which the iodide and buffer were added. The sample or measurement cell may be referred to as an electrochemical cell. An electrochemical cell may be comprised of a working electrode, auxiliary electrode, and a reference electrode (See FIG. 2). In an embodiment, the working electrode may be a conductive material. The working electrode may be a BDD working electrode. In an embodiment, the working electrode may be a carbon, platinum, BDD, or the like material.

In an embodiment, a BDD electrode may be used for measurement of formed iodine. BDD may have a low charging and/or background currents which may provide for low limits of detection. This low limit detection may be in contrast to a conventional electrode. BDD may also provide a large solvent potential window and/or fewer interferences as compared to gold or platinum. A BDD electrode may also provide a more robust, longer lasting, and less easily fouled electrode material. In an embodiment, the reduction of iodine and/or triiodide formed by the reaction of iodide with oxidants in a sample may be measured at the working electrode or the BDD working electrode (See FIG. 2).

In an embodiment, the reference electrode may be a silver-silver chloride, saturated calomel electrode (SCE) electrode, or the like. In an embodiment, the electrodes may utilize a range of fill solutions, gels, and frit constructions to best match the specific application and/or to optimize stability and longevity. In an embodiment, a pseudo reference electrode may be used such as a silver halide coated silver electrode or the like.

At 103, in an embodiment, the system and method may measure the amount of total oxidant in the seawater by measuring an electrochemical characteristic. In an embodiment the electrodes, as described above, may be disposed in a measurement cell of the measurement device. In an embodiment, a seawater sample may be placed with reagents. The reagents may include buffer and potassium iodide. In an embodiment, the measurement cell may have a stirring apparatus. For example, there may be a mechanical stirring paddle, arm, bar, or the like; or the stirring may be from the disturbance of fluid using a pump or the like. Additionally or alternatively, the measurement cell may have trapped particulates. The trapped particulates may be polymer beads. The trapped particulates may be moved about the measurement cell as a method to clean the cell and/or electrodes to remove buildup and fouling. In an embodiment, the cleaning cycle may use a wiper, brush, or the like to clean the measurement device.

In an embodiment, an electrochemical characteristic of the seawater solution may be measured. In an embodiment, a reduction of iodine and/or triiodide formed by a reaction of iodide with oxidant in a sample may be measured using electrochemical sweep methods. The sweep methods may include linear, cyclic voltammetry, or pulsed voltammetric methods. Additionally, or alternatively, amperometry may be used for measurement of reacted iodide to measure TRO. In an embodiment, a cleaning cycle may be performed using electrochemical methods.

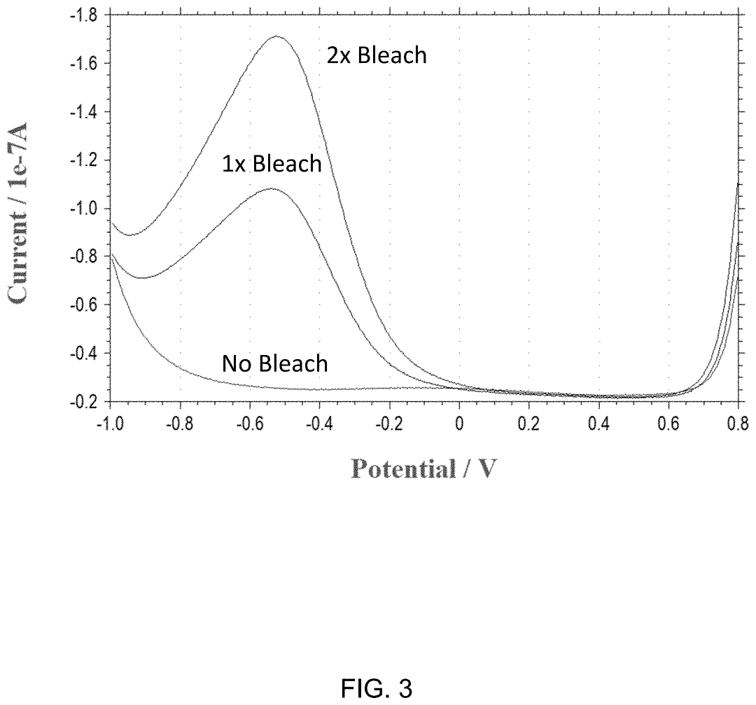

Referring to FIG. 3, in an embodiment a sample measurement of bleach at a BDD electrode is illustrated. In an embodiment, square wave voltammetry may be used to measure an amount of oxidant in a sample. The system and method may take a first measurement which may be referred to as a background measurement. The system and method may add reagents such as potassium iodide and a buffer. The system and method may measure iodine by electrochemical reduction. In an embodiment, the peak current correlates to an oxidant concentration in a sample. A voltage-current plot demonstrates an example embodiment for no bleach, lx bleach, and 2× bleach.

At 104, in an embodiment, the system and method may determine if an amount of oxidant may be measured. For example, an electrochemical reduction of iodine and/or triiodide formed by a reaction of iodide with oxidant in a sample may be measured using sweep methods. The sweep methods may include linear, cyclic voltammetry, or pulsed voltammetric methods. Additionally, or alternatively, amperometry may be used for measurement of reacted iodide to measure TRO. In an embodiment, a cleaning cycle may be performed using electrochemical methods. The measured electrochemical characteristics may be compared to expected values, historical values, or the like. For example, a system may output an unexpected value and request re-measurement of a seawater sample. In an embodiment, the system and method may be periodically testing using a known amount of oxidant in the sample. The system may then recalibrate or send an error report for maintenance.

Therefore, electrochemical characteristic measured from a seawater sample, of a solution containing oxidant or total chlorine may be correlated to the concentration of the oxidant or total chlorine in the aqueous solution. Voltage-current curves may be generated for a range of concentrations, for different samples, for any different condition that may affect measurement (e.g., temperature, sample content, turbidity, viscosity, measurement apparatus, aqueous sample chamber, etc.), or the like.

The oxidant measurement may be an output upon a device in the form of a display, printing, storage, audio, haptic feedback, or the like. Alternatively, or additionally, the output may be sent to another device through wired, wireless, fiber optic, Bluetooth®, near field communication, or the like. An embodiment may use an alarm to warn of a measurement or concentration outside acceptable levels. An embodiment may use a system to shut down water output or shunt water from sources with unacceptable levels of an analyte. For example, an analyte measuring device may use a relay coupled to an electrically actuated valve, or the like.

At 104, in an embodiment, if an amount of oxidant cannot be measured, the system may continue to measure total oxidant, alter electrochemical method protocols, and or obtain another sample. In an embodiment, the system and method may perform a cleaning cycle, assess reagent levels, recheck/calibrate with known standards, or the like. Additionally, or alternatively, the system may output an alarm, log an event, or the like.

If a concentration of total oxidant can be determined, the system may provide a measurement of total oxidant concentration at 105. The system may connect to a communication network. The system may alert a user or a network. This alert may occur whether a total oxidant measurement is determined or not. An alert may be in a form of audio, visual, data, storing the data to a memory device, sending the output through a connected or wireless system, printing the output or the like. The system may log information such as the measurement location, a corrective action, geographical location, time, date, number of measurement cycles, or the like. The alert or log may be automated, meaning the system may automatically output whether a correction was required or not. The system may also have associated alarms, limits, or predetermined thresholds. For example, if a total oxidant concentration reaches a threshold. Alarms or logs may be analyzed in real-time, stored for later use, or any combination thereof.

The various embodiments described herein thus represent a technical improvement to conventional oxidant or ORP measurement techniques. Using the techniques as described herein, an embodiment may use a BDD working electrode to measure oxidant in seawater. This is in contrast to DPD chemistry with limitations mentioned above. Such techniques provide a more robust and more accurate method for measuring oxidant in an aqueous or seawater solution. The various embodiments described herein thus represent a technical improvement to conventional ballast water treatment techniques. Using the techniques as described herein, an embodiment may use a method and device to measure total residual oxidant (TRO) in ballast water and measure oxidant levels. This is in contrast to conventional methods with limitations mentioned above. Such techniques provide a better method to treat ballast water and reduce levels of TRO to a de-ballast port.

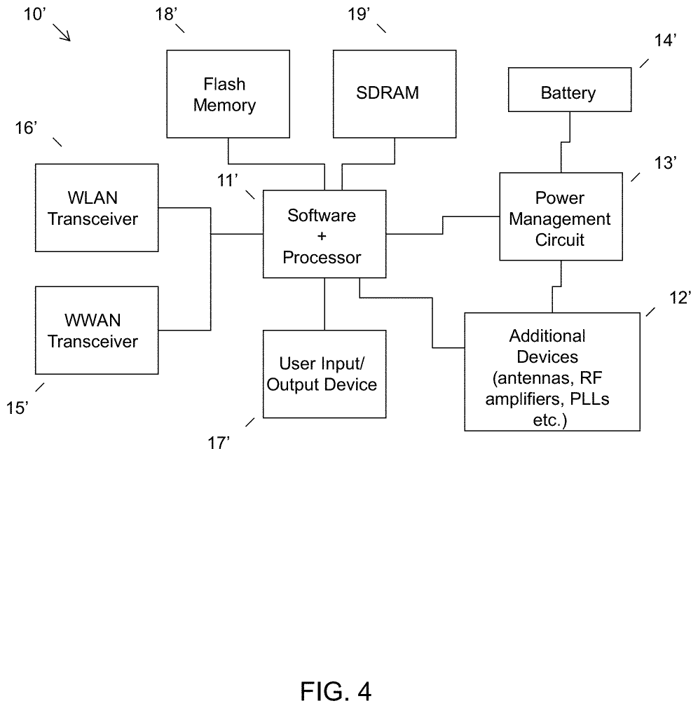

While various other circuits, circuitry or components may be utilized in information handling devices, with regard to an instrument for total oxidant measurement in seawater according to any one of the various embodiments described herein, an example is illustrated in FIG. 4. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment to perform total oxidant measurement of a sample of seawater.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for measuring total oxidant in a seawater sample, comprising:
forming a seawater solution and a formed iodine by introducing a buffer and an iodide reagent to a seawater sample, wherein the seawater sample contains an amount of oxidant;
placing the seawater solution in a measurement device, wherein the measurement device comprises a boron doped diamond working electrode reacting with the seawater solution and the formed iodine, wherein an electrochemical process reduces the formed iodine to iodide; and
measuring the amount of total oxidant in the seawater sample by measuring, using the measurement device, an amount of iodide in the seawater sample.

2. The method of claim 1, wherein the iodide reagent comprises potassium iodide.

3. The method of claim 1, wherein the buffer comprises an acid.

4. The method of claim 1, wherein the boron doped diamond working electrode reacts electrochemically with the formed iodine.

5. The method of claim 1, wherein the measurement device comprises a three-electrode electrochemical cell, one of the three electrodes being the boron doped diamond working electrode.

6. The method of claim 1, wherein the formed iodine reacts at the boron doped diamond working electrode via an electrochemical reduction.

7. The method of claim 1, further comprising a cleaning cycle, the cleaning cycle comprising at least one of: an electrochemical cleaning and a mechanical cleaning.

8. The method of claim 1, wherein the total oxidant is proportional to a current produced by the measurement device through the seawater sample.

9. The method of claim 1, wherein the total oxidant is measured using square wave voltammetry.

10. The method of claim 1, wherein the seawater sample comprises seawater from a ship ballast system.

11. A measurement device for measuring total oxidant in a seawater sample, comprising:
at least one measurement chamber;
a processor; and
a memory storing instructions executable by the processor to:
form a seawater solution and a formed iodine by introducing a buffer and an iodide reagent to a seawater sample, wherein the seawater sample contains an amount of oxidant;
place the seawater solution in a measurement device, wherein the measurement device comprises a boron doped diamond working electrode reacting with the seawater solution and the formed iodine, wherein an electrochemical process reduces the formed iodine to iodide; and
measure the amount of total oxidant in the seawater sample by measuring, using the measurement device, an amount of iodide in the seawater sample.

12. The device of claim 11, wherein the iodine reagent comprises potassium iodide.

13. The device of claim 11, wherein the buffer comprises an acid.

14. The device of claim 11, wherein the boron doped diamond working electrode reacts electrochemically with the formed iodine.

15. The device of claim 11, wherein the measurement device comprises a three-electrode electrochemical cell, one of the three electrodes being the boron doped diamond working electrode.

16. The device of claim 11, wherein the formed iodine reacts at the boron doped diamond working electrode via an electrochemical reduction.

17. The device of claim 11, further comprising a cleaning cycle, the cleaning cycle comprising at least one of: an electrochemical cleaning and a mechanical cleaning.

18. The device of claim 11, wherein the total oxidant is proportional to a current produced by the measurement device through the seawater sample.

19. The device of claim 11, wherein the total oxidant is measured using square wave voltammetry.

20. A product for measuring total oxidant in a seawater sample, comprising:
a storage device having code stored therewith, the code being executable by the processor and comprising:
code that forms a seawater solution and a formed iodine by introducing a buffer and an iodide reagent to a seawater sample, wherein the seawater sample contains an amount of oxidant;
code that places the seawater solution in a measurement device, wherein the measurement device comprises a boron doped diamond working electrode reacting with the seawater solution and the formed iodine, wherein an electrochemical process reduces the formed iodine to iodide; and
code that measures the amount of total oxidant in the seawater sample by measuring, using the measurement device, an amount of iodide in the seawater sample.
